(19) United States
(12) Patent Application Publication
(10) Pub. No.: US 2013/0281708 A1
(43) Pub. Date: Oct. 24, 2013

(54) Process for the Production of 2-Amino-5-Fluorothiazole

(71) Applicant: Matthew Neil Mattson, Santa Clara, CA (US)

(72) Inventor: Matthew Neil Mattson, Santa Clara, CA (US)

(73) Assignee: TAKEDA CALIFORNIA, INC., San Diego, CA (US)

(21) Appl. No.: 13/635,881

(22) PCT Filed: Mar. 4, 2011
(86) PCT No.: PCT/US11/27121

§ 371 Date: Jun. 20, 2013

Related U.S. Application Data
(60) Provisional application No. 61/315,250, filed on Mar. 18, 2010.

(51) Int. Cl. C07D 277/40; C07D 413/04; C07D 277/46; C07D 277/52; C07D 277/48; C07F 9/6539

(52) U.S. Cl. CPC C07D 277/40; C07D 277/48; C07F 9/6539; C07D 277/46; C07D 277/52; C07D 413/04
USPC 548/115; 548/199; 548/196; 548/195; 548/197; 548/181

(57) ABSTRACT

A process for the production of fluorinated compound represented by the formula (I):

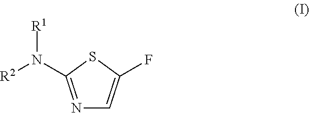

or salts thereof wherein R¹ and R² are the same or different and each is selected from the group consisting of a hydrogen atom, a carbonyl group, a sulfonyl group and a phosphoryl group.

### FIELD OF THE INVENTION

The present invention relates to a process for the production of fluorinated compound, and more particularly to a process for the production of 2-amino-5-fluorothiazole.

### BACKGROUND OF THE INVENTION

2-Amino-5-fluorothiazole and its derivatives are compounds useful for the production of various chemical compounds. For example, 2-amino-5-halothiazole is disclosed in US4086240 as an intermediate for the synthesis of herbicides. Also 2-amino-5-fluorothiazole is disclosed in WO2005/103021 in the process of producing modulators of glucokinase.

Certain processes for preparing 2-amino-5-fluorothiazole are disclosed in PCT/US04/03968 and PCT/GB2005/003170. However, these processes still suffer from the disadvantage of multi-step preparation or insufficient yield for the bulk synthesis.

### SUMMARY OF THE INVENTION

The present invention provides an improved, convenient and highly-efficient process for the production of substituted or unsubstituted 2-amino-5-fluoro thiazole or a salt thereof. More specifically, the present invention relates to:

(1) A method for producing a compound represented by the formula (I):

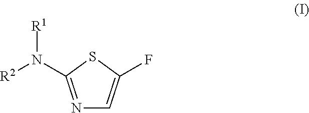

wherein R¹ and R² are the same or different and each is selected from the group consisting of a hydrogen atom, an optionally substituted carbonyl group, an optionally substituted sulfonyl group and an optionally substituted phosphoryl group, or a salt thereof comprising reacting a compound represent by the formula (II):

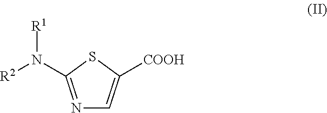

or a salt thereof with a fluorine donor.

(2) The method according to (1), wherein each of R¹ and R² is a hydrogen atom.

(3) The method according to (1), wherein R¹ is a hydrogen atom and R² is an optionally substituted carbonyl group which forms a carbamate with the adjacent nitrogen.

(4) The method according to (1), wherein R¹ is a hydrogen atom and R² is an optionally substituted carbonyl group which forms an amide with the adjacent nitrogen.

(5) The method according to (1), wherein each of R¹ and R² is an optionally substituted carbonyl group which forms an amide with the adjacent nitrogen.

(6) The method according to (5), wherein R¹ and R² are taken together to form isoindoline 1,3-dione.

(7) The method according to (1), wherein R¹ and R² are the same or different and each is selected from the group consisting of a hydrogen atom, an optionally substituted C₁₋₆ alkoxy-carbonyl, an optionally substituted C₁₋₆ alkyl-carbonyl, an optionally substituted sulfonyl, and an optionally substituted phosphoryl.

(8) The method according to (1), wherein R¹ and R² are the same or different and each is selected from the group consisting of a hydrogen atom, tert-butoxycarbonyl, benzyloxycarbonyl, methylcarbonyl, diphenoxyphosphoryl, and 4-methylphenylsulfonyl.

(9) The method according to (1), wherein R¹ is a hydrogen and R² is C₁₋₆ alkyl-carbonyl which is substituted by optionally substituted phenyl.

(10) The method according to (7)-(9), wherein R¹ is a hydrogen atom and R² is not a hydrogen atom.

(11) The method according to (1), wherein the fluorine donor is 1-Chloromethyl-4-fluoro-1,4-diazoniabicyclo[2.2.2]octane bis(tetrafluoroborate).

(12) The method according to (1), wherein the fluorine donor is 1-fluoro-4-hydroxy-1,4-diazoniabicyclo[2.2.2]octane bis(tetrafluoroborate).

(13) The method according to (1), wherein the compound represented by the formula (II) is produced by hydrolysis of an alkyl ester thereof.

(14) The method according to (12), wherein the alkyl ester is methyl ester.

(15) A method for producing a compound represented by the formula (III):

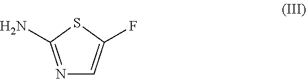

or a salt thereof comprising the steps of:
(a) reacting a compound represent by the formula (IIa):

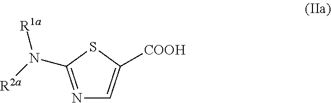

or a salt thereof with a fluorine donor under conditions that form the compound represented by the formula (I), wherein R^{1a} and R^{2a} are the same or different and each is selected from the group consisting of an optionally substituted carbonyl group, an optionally substituted sulfonyl group and an optionally substituted phosphoryl group, and
(b) deprotecting the amine of formula (I) to produce the compound of formula (III).

(16) A process for producing 2-amino-5-fluorothiazole characterized by reacting a compound of the formula (II) with a fluorine donor to give a compound of formula (I).

### DETAILED DESCRIPTION OF THE INVENTION

The present invention relates to a fluorination of 2-aminothiazole-5-carboxylic acid whose amino group can be substituted by one or two substituent selected from the group consisting of a carbonyl group, a sulfonyl group and a phosphoryl group. The present invention provides high yield and is an attractive route to produce substituted or unsubstituted 2-amino-5-fluoro thiazole.

The general sequence from substituted or unsubstituted methyl 2-aminothiazole-5-carboxylate is described in Scheme 1-4.

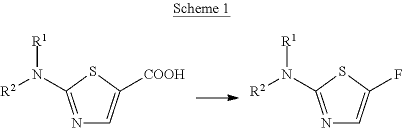

Wherein R¹ and R² are the same or different and each is selected from the group consisting of a hydrogen atom, an optionally substituted carbonyl group, an optionally substituted sulfonyl group and an optionally substituted phosphoryl group.

In one embodiment, each of R¹ and R² is a hydrogen atom.

In another embodiment, R¹ is a hydrogen atom and R² is selected from the group consisting of an optionally substituted carbonyl group, an optionally substituted sulfonyl group and an optionally substituted phosphoryl group.

As an optionally substituted carbonyl group, —CO—OR′ and —COR^{a}, wherein R^{a} is a hydrogen atom, an optionally substituted hydrocarbon or an optionally substituted heterocycle, and the like can be mentioned. As an optionally substituted sulfonyl group, —SO₂R^{a} wherein R^{a} is a hydrogen atom, an optionally substituted hydrocarbon or an optionally substituted heterocycle, and the like can be mentioned. As an optionally substituted phosphoryl group, —PO(OR^{a}) (OR^{b}), wherein R^{a} and R^{b} are the same or different and each is a hydrogen atom, an optionally substituted hydrocarbon or an optionally substituted heterocycle, and the like can be mentioned.

The carbonyl group can form a carbamate with the adjacent nitrogen. As the carbamate, for example, methyl carbamate, ethyl carbamate, 9-fluorenylmethyl carbamate (Fmoc), 9-(2-sulfo)fluorenylmethyl carbamate, 9-(2,7-dibromo)fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate, 1-(1-adamantyl)-1-methylethyl carbamate (Adpoc), 1,1-dimethyl-2-haloethyl carbamate, 1,1-dimethyl-2,2-dibromoethyl carbamate (DB-t-Boc), 1,1-dimethyl-2,2,2-trichloroethyl carbamate (TCBoc), 1-methyl-1-(4-biphenylyl)ethyl carbamate (Bpoc), 1-(3,4-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (t-Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate, 9-anthrylmethyl carbamate, and diphenylmethyl carbamate can be mentioned.

The carbonyl group can also form an amide with the adjacent nitrogen. As the amide, for example, formamide, acetamide, chloroacetamide, trichloroacetamide, trifluoroacetamide, trifluoroacetamide, phenylacetamide, 3-phenylpropanamide, picolinamide, 3-pyridylcarboxamide, benzamide, p-phenylbenzamide can be mentioned.

When both R¹ and R² are —COR^{a}, R¹ and R² are taken together with the adjacent nitrogen to form a ring system such as isoindoline 1,3-dione.

Preferably, R¹ and R² are the same or different and each is selected from the group consisting of a hydrogen atom, an optionally substituted C₁₋₆ alkoxy-carbonyl, an optionally substituted C₁₋₆ alkyl-carbonyl, an optionally substituted sulfonyl, and an optionally substituted phosphoryl. C₁₋₆ alkyl, carboxyl, optionally substituted phenyl, and phenoxy can be mentioned as the substituent of the C₁₋₆ alkoxy-carbonyl, the C₁₋₆ alkyl-carbonyl, the sulfonyl, and the phosphoryl. In another embodiment, R¹ and R² are the same or different and each is selected from the group consisting of a hydrogen atom, tert-butoxycarbonyl, benzyloxycarbonyl, methylcarbonyl, diphenoxyphosphoryl, or 4-methylphenylsulfonyl. In another embodiment, R¹ and R² are the same or different and each is selected from the group consisting of a hydrogen atom, tert-butoxycarbonyl, benzyloxycarbonyl, or methylcarbonyl. In another embodiment, R¹ is a hydrogen and R² is C₁₋₆ alkyl-carbonyl which is substituted by optionally substituted phenyl. In another embodiment, R¹ is a hydrogen and R² is benzylcarbonyl optionally substituted by halogen, C₁₋₆ alkyl, and C₁₋₆ alkoxy.

When R¹ or R² are not hydrogen, they can be removed following the fluorination, using any of a number of chemical reactions known to a person skilled in the art. The chemical reactions comprise, for example but not limited to, acidic hydrolysis, catalytic hydrogenolysis, elimination, deacylation and isomerization. A suitable solvent for each reaction can be chosen by a person skilled in the art, for example but not limited to, dilute acetic acid, hydrochloric acid, iodotrimethylsilane, aluminium chloride, triethylsilane, zinc in acetic acid, trifluoroacetic acid, Pd, ammonia, metal alkoxides, metal hydroxides, and metal carbonates. These conditions will vary depending on the nature of R¹ and R².

As used herein, the “fluorine donor” is an agent which can provide the fluorination process with F⁺. For example, a fluorine donor is an electrophilic fluorination reagents such as:

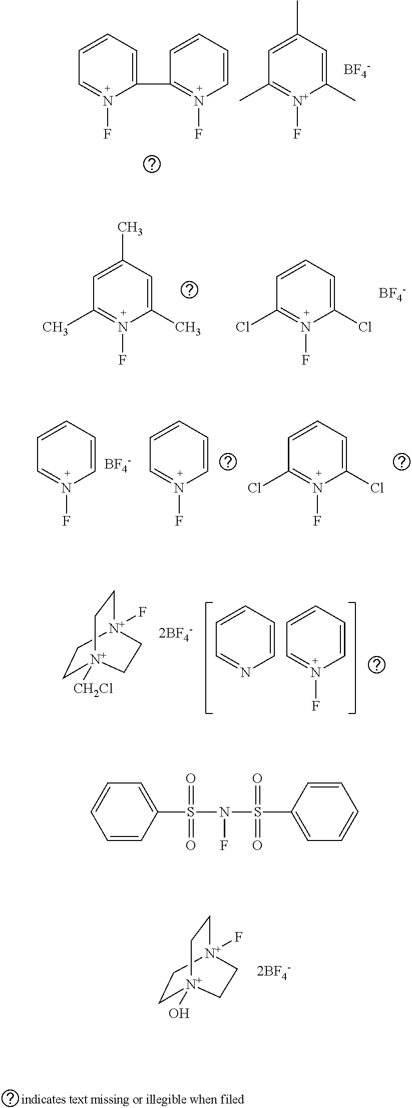

As a preferable fluorine donor, 1-Chloromethyl-4-fluoro-1,4-diazoniabicyclo[2.2.2]octane bis(tetrafluoroborate) (Selectfluor®) can be mentioned. As another preferable fluorine donor, 1-fluoro-4-hydroxy-1,4-diazoniabicyclo[2.2.2]octane bis(tetrafluoroborate) can also be mentioned.

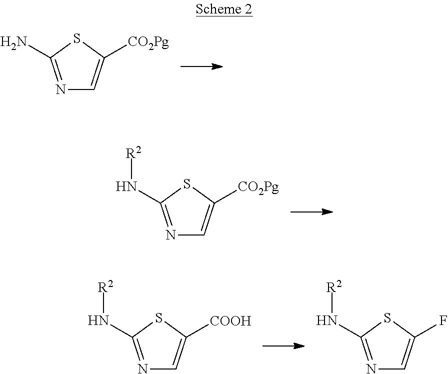

Both of the amino group and the carboxyl group are protected in this scheme. Wherein, R² is selected from the group consisting of an optionally substituted carbonyl group, an optionally substituted sulfonyl group and an optionally substituted phosphoryl group. Pg is a suitable protecting group. As a preferable protecting group, C1-6 alkyl can be mentioned. As a more preferable protecting group, methyl can be mentioned. Methyl 2-aminothiazole-5-carboxylate is commercially available for the starting material.

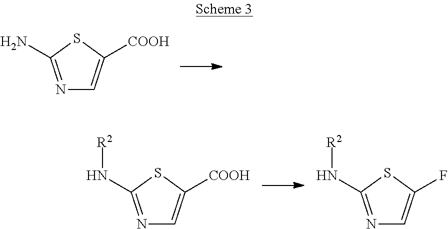

Wherein R² is selected from the group consisting of an optionally substituted carbonyl group, an optionally substituted sulfonyl group and an optionally substituted phosphoryl group. The starting material, 2-aminothiazole-5-carboxylate, is commercially available.

In Scheme 2 and Scheme 3, R² can be removed following the fluorination, if necessary for use of the final product, using any of a number of chemical reactions known to a person skilled in the art. The chemical reactions comprise, for example but not limited to, acidic hydrolysis, catalytic hydrogenolysis, elimination, deacylation and isomerization. A suitable solvent for each reaction can be chosen by a person skilled in the art, for example but not limited to, dilute acetic acid, hydrochloric acid, iodotrimethylsilane, aluminium chloride, triethylsilane, zinc in acetic acid, trifluoroacetic acid, Pd, ammonia, metal alkoxides, metal hydroxides, and metal carbonates. These conditions will vary depending on the nature of R².

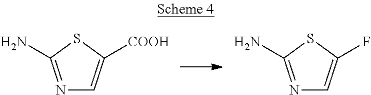 [SMILES: Nc1ncc(C(=O)O)s1 ; Nc1ncc(F)s1]

2-Amino-thiazole-5-carboxylic acid is directly fluorinated to 5-Fluoro-thiazol-2-ylamine in this scheme.

For the fluorination process in Scheme 2-4, the fluorine donors mentioned in Scheme 1 can also be used in this scheme.

### EXAMPLES

NMR spectra were recorded in DMSO-d6 at 200 MHz.

HPLC method TKD-1 Agilent 1100/1200, Zorbax SB-C8, 4.6×100 mm, 3.5 micron, 35° C. column temperature, 254 nm detection, 1.20 mL/min, C=0.1% TFA in CH₃CN, D=0.1% TFA in H₂O, flow as per Table 1.

TABLE 1
Gradient flow for TKD-1

| Time (min) | % C | % D |
|---|---|---|
| 0 | 5 | 95 |
| 15 | 90 | 10 |
| 16 | 90 | 10 |
| 17 | 5 | 95 |

Molecular weights were confirmed by HPLC-MS using an Applied Biosystems API-150EX mass spectrometer in with an ABI “Ion Sprayer” electrospray ionization source (ESI) operated in the positive ion mode (100-900 amu scan range). A Shimadzu VP binary (LC10AD pumps) high-pressure mixed gradient HPLC system equipped with a dual wavelength UV-Vis detector (SPD-10A), a CTC/Leap Technologies HTC PAL autosampler, and a Sedex model 75 evaporative light scattering detector (ELSD). Eluent A was 0.1% trifluoroacetic acid in HPLC grade water and eluent B was 0.1% trifluoroacetic acid in HPLC grade acetonitrile. A linear gradient from 5% B to 100% B over 10 minutes at a flow rate of 1.5 mL/min was used. The HPLC column was a Phenomenex Onyx monolithic C18, 50×4.6 mm (P/N CHO-7644).

### Preparation 1

### 2-(tert-Butoxycarbonylamino)thiazole-5-carboxylic acid

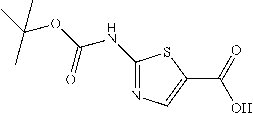 [SMILES: CC(C)(C)OC(=O)Nc1ncc(C(=O)O)s1]

2-Amino-thiazole-5-carboxylic acid methyl ester (4.0 g, 25.28 mmol) was suspended in THF (100 ml). Di-tert-butyl dicarbonate (6.63 g, 30.34 mmol) was added to the reaction vessel and the mixture was stirred vigorously. Next, Triethylamine (7.05 mL, 50.57 mmol) and 4-Dimethylaminopyridine (316 mg, 2.53 mmol) were added to the reaction. The reaction was stirred at room temperature for 16 hours. A brown precipitate was present upon completion of the reaction. The reaction mixture was concentrated down in vacuo and dried in a vacuum oven to afford the crude product, 2-tert-Butoxycarbonylamino-thiazole-5-carboxylic acid methyl ester (6.5 g, 100% yield), which was then taken on to subsequent reaction. LCMS [M+H] 258.9.

2-tert-Butoxycarbonylamino-thiazole-5-carboxylic acid methyl ester (6.5 g, 25.28 mmol) was dissolved in THF (150 mL) and Methanol (200 mL) and placed in a 60° C. oil bath, while stirring vigorously. Sodium hydroxide (5.1 g, 127.5 mmol) was dissolved in water (40 mL) and slowly added to the reaction vessel. The reaction was stirred for 16 hours at 60° C. After this time, the reaction mixture was concentrated in vacuo to about ⅓ the original volume. Water (60 mL) was added to the reaction mixture and the solution was acidified to pH=1-2 with 3M HCl (aq) to afford the product, 2-tert-Butoxycarbonylamino-thiazole-5-carboxylic acid, as a white precipitate. The product was washed with water and taken on to the next reaction as is. LCMS [M+H] 244.9.

### Preparation 2

### Methyl 2-(benzyloxycarbonylamino)thiazole-5-carboxylate

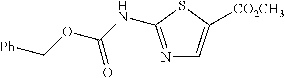 [SMILES: COC(=O)c1cnc(NC(=O)OCc2ccccc2)s1]

Methyl 2-aminothiazole-5-carboxylate, 1.00 g, 6.3 mmol, was added to a 50 mL, 3-necked round-bottomed flask fitted with a mechanical agitator. Tetrahydrofuran, 15 mL, was added followed by diisopropyl ethylamine, 1.7 mL, 1.5 eq. Benzyl chloroformate, 1.1 mL, 1.25 eq., was added which caused the temperature to rise to about 45° C. After stirring overnight, 10 mL of water and 5 mL of methanol was added and the mixture was stirred at ambient temperature for two hours. The product was isolated by filtration, was rinsed with aqueous methanol and then water. Drying in the air provided 1.79 g, 97%, of an off-white solid, LC/MS: 293.2, NMR: 12.2 (s, 1H), 8.08 (s, 1H), 7.45-7.35 (m, 5H), 5.27 (s, 2H), 3.80 (s, 3H); HPLC: RT 9.83 min, 100%.

### Preparation 3

### 2-(Benzyloxycarbonylamino)thiazole-5-carboxylic acid

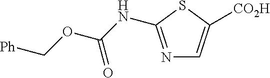 [SMILES: O=C(Nc1ncc(C(=O)O)s1)OCc1ccccc1]

Methyl 2-(benzyloxycarbonylamino)thiazole-5-carboxylate, 1.73 g, 5.9 mmol, was combined in a 100 mL, 3-necked round-bottomed flask fitted with a mechanical agitator with 15 mL of methanol. Potassium hydroxide, 1.1 g of 90%, 3 eq., was dissolved in 5 mL of water and this solution was added to the reaction. After three hours at ambient temperature, 10 mL of water was added and the pH was adjusted to 10 by addition of dilute hydrochloric acid and the mixture was washed twice with methylene chloride. The mixture was diluted with 15 mL of water and 10 mL of methanol and the pH was lowered to 3 with dilute hydrochloric acid. The resulting slurry was stirred overnight at ambient temperature. The product was isolated by filtration, was rinsed with aqueous methanol and water, and dried under high vacuum to afford 1.19 g, 72% of solid product, LC/MS: 279.2; NMR: 12.9 (s, 1H), 7.99 (s, 1H), 7.40 (m, 5H), 5.27 (s, 2H); HPLC: RT 7.92 min, 96.1%.

### Preparation 4

### Methyl 2-(diphenoxyphosphorylamino)thiazole-5-carboxylate

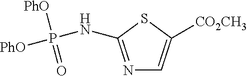 [SMILES: COC(=O)c1cnc(NP(=O)(Oc2ccccc2)Oc2ccccc2)s1]

Methyl 2-aminothiazole-5-carboxylate, 1.0 g, 6.20 mmol, was dissolved in 4-methylmorpholine, 20 mL, and cooled in an ice bath for 15 minutes. Diphenyl chlorophosphate, 4 mL, 18.6 mmol, was added dropwise. The ice bath was removed and the reaction was allowed to stir at room temperature overnight. The reaction mixture was concentrated in vacuo and the residue was dissolved in methylene chloride, 50 mL. The solution was washed with saturated aqueous NaHCO₃, 1M HCl, and brine, respectively. During the brine wash, a precipitate began to form in the organic layer. The organic layer was separated and its volume was reduced in half. The solid was filtered off and dried in vacuo to afford the white product, 1.6 g, 65%, LC/MS 391.1; NMR 13.0 (broad s, 1H), 8.01 (s, 1H), 7.38 (m, 4H), 7.21 (m, 6H), 3.77 (s, 3H); HPLC: RT 9.64 min, 98.7%.

### Preparation 5

### 2-(Diphenoxyphosphorylamino)thiazole-5-carboxylic acid

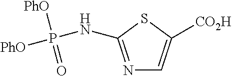 [SMILES: O=C(O)c1cnc(NP(=O)(Oc2ccccc2)Oc2ccccc2)s1]

Methyl 2-(diphenoxyphosphorylamino)thiazole-5-carboxylate, 1.55 g, 4.0 mmol, was combined in a 100 mL, 3-necked round-bottomed flask fitted with a mechanical agitator with 10 mL of methanol. Potassium hydroxide, 0.74 g of 90%, 3 eq., was dissolved in 5 mL of water and this solution was added to the reaction. After stirring overnight at ambient temperature, 10 mL of water and 10 mL of methanol was added and the pH was adjusted to 2 by addition of dilute hydrochloric acid. The resulting slurry was stirred for two hours at ambient temperature. The product was isolated by filtration, was rinsed with aqueous methanol and water, and dried under high vacuum at ambient temperature to afford 1.40 g, 94% of white solid, LC/MS: 376.9; NMR: 7.89 (s, 1H), 7.39 (m, 4H), 7.19 (m, 6H); HPLC: RT 8.25 min, 98.6%.

### Preparation 6

### Methyl 2-acetamidothiazole-5-carboxylate

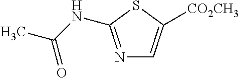

Methyl 2-aminothiazole-5-carboxylate, 1.58 g, 10 mmol, was added to a 50 mL, 3-necked round-bottomed flask fitted with a mechanical agitator. Tetrahydrofuran, 15 mL, was added followed by triethylamine, 2.1 mL, 1.5 eq. Acetic anhydride, 1.3 mL, 1.4 eq., was added. After stirring overnight, 20 mL of water and 5 mL of methanol was added and the mixture was stirred at ambient temperature for two hours. The product was isolated by filtration, was rinsed with aqueous methanol and then water. Drying in the air provided 1.81 g, 90%, of an off-white solid, LC/MS: 201.1; NMR: 12.6 (s, 1H), 8.15 (s, 1H), 3.81 (s, 3H), 2.19 (s, 3H); HPLC: RT 5.83 min, 99.5%.

### Preparation 7

### 2-Acetamidothiazole-5-carboxylic acid

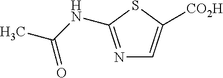

Methyl 2-acetamidothiazole-5-carboxylate, 1.74 g, 8.7 mmol, was combined in a 100 mL, 3-necked round-bottomed flask fitted with a mechanical agitator with 8 mL of methanol. Potassium hydroxide, 1.6 g of 90%, 3 eq., was dissolved in 8 mL of water and this solution was added to the reaction. After two hours at ambient temperature, the solution was washed with 20 mL of methylene chloride. The pH of the aqueous phase was adjusted to 3 by the addition of dilute hydrochloric acid. The resulting slurry was stirred overnight at ambient temperature. The product was isolated by filtration, was rinsed with aqueous methanol and water, and dried under high vacuum at ambient temperature to afford 1.55 g, 96% of white solid, LC/MS: 186.9, NMR: 13.1 (s, 1H), 12.51 (s, 1H), 8.05 (s, 1H), 2.18 (s, 3H); HPLC: RT 3.99 min, 94.5%.

### Preparation 8

### Methyl 2-(4-methylphenylsulfonamido)thiazole-5-carboxylate

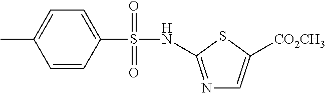 [SMILES: COC(=O)c1cnc(NS(=O)(=O)c2ccc(C)cc2)s1]

Methyl 2-aminothiazole-5-carboxylate, 1.00 g, 6.3 mmol, was added to a 50 mL, 3-necked round-bottomed flask fitted with a mechanical agitator. Tetrahydrofuran, 10 mL, was added followed by 4-methylbenzenesulfonyl chloride, 1.81 g, 1.5 eq., then diisopropyl ethylamine, 2.0 mL, 1.8 eq. 4-Dimethylaminopyridine, 0.08 g, 0.1 eq., was added and the mixture was stirred at ambient temperature for five days. After evaporation of most of the solvent at ambient temperature, 20 mL of methanol was added followed by 15 mL of water, and the mixture was stirred at ambient temperature for one hour. The product was isolated by filtration and was rinsed with water. Drying under high vacuum at ambient temperature provided 0.82 g, 42%, of an off-white solid, LC/MS: 313.0, NMR: 8.16 (s, 1H), 7.72 (d, 2H), 7.37 (d, 2H), 3.79 (s, 3H), 2.36 (s, 3H); HPLC: RT 8.38 min, 99.0%.

### Preparation 9

### 2-(4-Methylphenylsulfonamido)thiazole-5-carboxylic acid

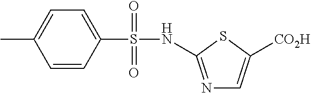 [SMILES: Cc1ccc(S(=O)(=O)Nc2ncc(C(=O)O)s2)cc1]

Methyl 2-(4-methylphenylsulfonamido)thiazole-5-carboxylate, 0.79 g, 2.5 mmol, was combined in a 100 mL, 3-necked round-bottomed flask fitted with a mechanical agitator with 10 mL of methanol. Potassium hydroxide, 0.47 g of 90%, 3 eq., was dissolved in 5 mL of water and this solution was added to the reaction. After two hours at ambient temperature, another 0.16 g of KOH was added and the mixture was stirred at 50° C. for two hours. After cooling to ambient temperature, the pH was adjusted to 2 by the addition of dilute hydrochloric acid. The resulting slurry was stirred for 30 minutes at ambient temperature. The product was isolated by filtration, was rinsed with aqueous methanol and water, and dried under high vacuum at ambient temperature to afford 0.63 g, 84%, of a tan solid, LC/MS: 299.1; NMR: 8.01 (s, 1H), 7.71 (d, 2H), 7.36 (d, 2H), 2.36 (s, 3H); HPLC: RT 6.70 min, 99.6%.

### Preparation 10

### Methyl 2-(2-(methoxycarbonyl)benzamido)thiazole-5-carboxylate

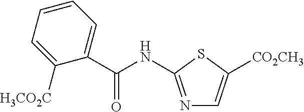 [SMILES: COC(=O)c1cnc(NC(=O)c2ccccc2C(=O)OC)s1]

Methyl 2-aminothiazole-5-carboxylate, 1.00 g, 6.3 mmol, was added to a 50 mL, 3-necked round-bottomed flask fitted with a mechanical agitator. Tetrahydrofuran, 15 mL, was added followed by diisopropyl ethylamine, 2.8 mL, 2.5 eq. o-Phthaloyl dichloride, 1.1 mL, 1.2 eq., was added which caused the temperature to rise to about 55° C. After stirring at ambient temperature for an hour, 5 mL of methanol and 10 mL of water was added and the mixture was stirred at ambient temperature for an hour. The product was isolated by filtration, was rinsed with aqueous methanol and then water. A second crop was collected from the mother liquor; it was filtered off and rinsed with water. The combined solids were dried under high vacuum to provide 1.36 g of an off-white solid. LC/MS showed two peaks, m/z=321.2 and 162.9 for the first and 293.2 for the second. NMR: 13.1 (s, 1H), 8.22 (s, 1H), 7.94 (m, 1H), 7.70 (m, 3H), 3.84 (s, 3H), 3.76 (s, 3H); HPLC: RT 8.42 min, 99%.

### Preparation 11

### 2-(2-Carboxybenzamido)thiazole-5-carboxylic acid

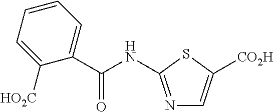 [SMILES: O=C(O)c1cnc(NC(=O)c2ccccc2C(=O)O)s1]

Methyl 2-(2-(methoxycarbonyl)benzamido)thiazole-5-carboxylate, 1.28 g, 4 mmol, was combined in a 100 mL, 3-necked round-bottomed flask fitted with a mechanical agitator with 15 mL of methanol. Potassium hydroxide, 0.83 g of 90%, 3.3 eq., was dissolved in 5 mL of water and this solution was added to the reaction. After stirring overnight at ambient temperature, another 0.29 g of KOH was added and the mixture was stirred at 50° C. for 2.5 hours. The mixture was cooled and the pH of the aqueous phase was adjusted to 3 by the addition of dilute hydrochloric acid. The resulting slurry was stirred for 30 minutes at ambient temperature. The product was isolated by filtration, was rinsed with aqueous methanol and water, and dried under high vacuum at ambient temperature to afford 1.09 g, 89% of white solid, LC/MS: 149.1, 275.1, and 292.9; NMR: 13.1 (broad s, 1H), 13.0 (broad s, 1H), 8.10 (s, 1H), 7.93 (m, 1H), 7.64 (m, 3H); HPLC: RT 5.41 min, 99.2%.

### Example 1

### Synthesis of 5-Fluoro-thiazol-2-ylamine

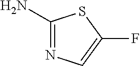

2-Amino-thiazole-5-carboxylic acid (2.16 g, 15 mmol) and KHCO₃ (5.25 g, 52.5 mmol) were suspended in a mixture of water (7 ml), methanol (30 ml), dioxane (42 ml) and Toluene (42 ml). This was stirred at room temperature for 2 hours. Next, F-TEDA (9.3 g, 26.25 mmol) was added to the reaction vessel. This mixture was allowed to stir at room temperature for 1 hour. After this period, the reaction mixture was filtered and the filtrate was concentrated in vacuo to afford a dark residue. This material was purified by silica flash chromatography (DCM:THF, 8:2) to afford the product, 5-Fluoro-thiazol-2-ylamine (560 mg, 31.6%).

2-Amino-thiazole-5-carboxylic acid (4.32 g, 30 mmol) and K₂HPO₄ (20 g, 120 mmol) were suspended in a mixture of methanol (40 ml), dioxane (70 ml) and Toluene (70 ml). This was stirred at room temperature for 2 hours. Next, F-TEDA (17 g, 6.91 mmol) was added to the reaction vessel. This mixture was allowed to stir at room temperature for 30 minutes. After this period, the reaction mixture was filtered and the filtrate was put through a silica gel plug, acidified with HCl in Dioxane, concentrated in vacuo and lyophilized for 12 hours to afford the product, 5-Fluoro-thiazol-2-ylamine HCl salt (2.55 g, 55%).

2-Amino-thiazole-5-carboxylic acid (8.64 g, 60 mmol) and K₃PO₄ (13.04 g, 90 mmol) were suspended in a mixture of methanol (100 ml), dioxane (150 ml) and toluene (150 ml). This was stirred at room temperature for 2 hours. Next, F-TEDA (34.5 g, 97 mmol) was added to the reaction vessel. This mixture was allowed to stir at room temperature for 30 minutes. After this period, the reaction mixture was filtered through a silica gel plug, acidified with HCl in dioxane, concentrated in vacuo and lyophilized for 12 hours to afford the product, 5-Fluoro-thiazol-2-ylamine HCl salt (6.42 g, 69%).

### Example 2

### Synthesis of tert-butyl 5-fluorothiazol-2-ylcarbamate

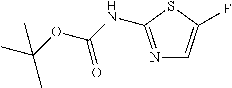

2-(tert-Butoxycarbonylamino)thiazole-5-carboxylic acid, 0.50 g, 2.0 mmol, and F-TEDA, N-chloromethyl-N′-fluorotriethylenediammonium bis(tetrafluoroborate), 1.16 g, 1.6 eq., were combined with 6 mL of methyl tetrahydrofuran in a 50 mL, 3-necked round-bottomed flask fitted with a mechanical agitator. Potassium phosphate, tribasic monohydrate, 1.30 g, 2.8 eq., was dissolved in 4 mL of water and this solution was added dropwise to the reaction over about 15 minutes while maintaining the reaction contents at 2-5° C. The ice bath was removed and the mixture was stirred vigorously at ambient temperature for three hours. Solids were removed, rinsed with methyl tetrahydrofuran, and discarded. The phases were separated and the aqueous layer was extracted with 10 mL of methyl tetrahydrofuran and the combined organic layers were washed with 15 mL of dilute brine. Evaporation of the solvent at ambient temperature provided 0.29 g, 66%, of a tan solid of tert-butyl 5-fluorothiazol-2-ylcarbamate, LC/MS: 219.3; NMR: 11.50 (broad s, 1H), 7.17 (d, J=2.2, 1H), 1.47 (s, 3H); HPLC: RT 9.60 min, 96.8%.

### Example 3

### Synthesis of benzyl 5-fluorothiazol-2-ylcarbamate

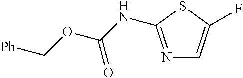 [SMILES: O=C(Nc1ncc(F)s1)OCc1ccccc1]

2-(Benzyloxycarbonylamino)thiazole-5-carboxylic acid, 0.58 g, 2.1 mmol, and F-TEDA, 1.18 g, 1.6 eq., were combined with 10 mL of methyl tetrahydrofuran in a 50 mL, 3-necked round-bottomed flask fitted with a mechanical agitator. Potassium phosphate, tribasic monohydrate, 1.35 g, 2.8 eq., was dissolved in 6 mL of water and this solution was added dropwise to the reaction over about 15 minutes while maintaining the reaction contents at 3-8° C. The ice bath was removed and the mixture was stirred vigorously at ambient temperature for three hours. Solids were removed, rinsed with methyl tetrahydrofuran, and discarded. The phases were separated and the aqueous layer was extracted with 10 mL of methyl tetrahydrofuran and the combined organic layers were washed with 15 mL of water. Evaporation of the solvent provided 0.45 g, 85%, of an off-white solid of benzyl 5-fluorothiazol-2-ylcarbamate, LC/MS: 253.2; NMR: 11.92 (broad s, 1H), 7.42 (m, 5H), 7.20 (d, J=2.2, 1H), 5.22 (s, 2H); HPLC: RT 10.02 min, 95.4%.

### Example 4

### Synthesis of diphenyl 5-fluorothiazol-2-ylphosphoramidate

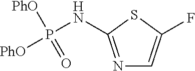 [SMILES: O=P(Nc1ncc(F)s1)(Oc1ccccc1)Oc1ccccc1]

2-(Diphenoxyphosphorylamino)thiazole-5-carboxylic acid, 0.89 g, 2.4 mmol, and F-TEDA, 1.34 g, 1.6 eq., were combined with 10 mL of methyl tetrahydrofuran in a 50 mL, 3-necked round-bottomed flask fitted with a mechanical agitator. Potassium phosphate, tribasic monohydrate, 1.55 g, 2.8 eq., was dissolved in 6 mL of water and this solution was added dropwise to the reaction over about 15 minutes while maintaining the reaction contents at 2-5° C. The ice bath was removed and the mixture was stirred vigorously at ambient temperature for four hours. Solids were removed, rinsed with methyl tetrahydrofuran, and discarded. The phases were separated and the organic layer was washed with 15 mL of dilute brine. Evaporation of the solvent provided 0.66 g of a red-brown foam as the expected product, diphenyl 5-fluorothiazol-2-ylphosphoramidate.

### Example 5

### Synthesis of N-(5-fluorothiazol-2-yl) acetamide

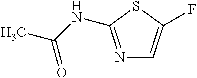

2-Acetamidothiazole-5-carboxylic acid, 0.83 g, 4.5 mmol, and F-TEDA, 2.53 g, 1.6 eq., were combined with 10 mL of methyl tetrahydrofuran in a 50 mL, 3-necked round-bottomed flask fitted with a mechanical agitator. Potassium phosphate, tribasic monohydrate, 2.90 g, 2.8 eq., was dissolved in 10 mL of water and this solution was added dropwise to the reaction over 20 minutes while maintaining the reaction contents at 3-8° C. The ice bath was removed and the mixture was stirred vigorously at ambient temperature for three hours. Solids were removed, rinsed with methyl tetrahydrofuran, and discarded. The phases were separated and the aqueous layer was extracted twice with 10 mL of methyl tetrahydrofuran each and the combined organic layers were washed with 15 mL of dilute brine. Evaporation of the solvent and drying in air provided 0.35 g, 49%, of an off-white solid of N-(5-fluorothiazol-2-yl) acetamide, LC/MS: 161.3; NMR: 12.15 (broad s, 1H), 7.27 (d, J=2.0, 1H), 2.12 (s, 3H); HPLC: RT 5.40 min, 96.4%.

### Example 6

### Synthesis of 2-(4-methylphenylsulfonamido) thiazole-5-carboxylic acid

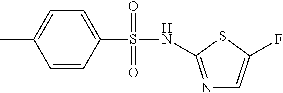 [SMILES: Cc1ccc(S(=O)(=O)Nc2ncc(F)s2)cc1]

2-(4-Methylphenylsulfonamido)thiazole-5-carboxylic acid, 0.66 g, 2.2 mmol, and F-TEDA, 1.25 g, 1.6 eq., were combined with 10 mL of methyl tetrahydrofuran in a 50 mL, 3-necked round-bottomed flask fitted with a mechanical agitator. Potassium phosphate, tribasic monohydrate, 1.42 g, 2.8 eq., was dissolved in 6 mL of water and this solution was added dropwise to the reaction over 15 minutes while maintaining the reaction contents at 3-8° C. The ice bath was removed and the mixture was stirred vigorously at ambient temperature for four hours. Solids were removed, rinsed with methyl tetrahydrofuran, and discarded. The phases were separated and the aqueous layer was extracted with 10 mL of methyl tetrahydrofuran and the combined organic layers were washed with 15 mL of water. Evaporation of the solvent provided 0.49 g of a tan foam. HPLC analysis indicated the presence of three major products, with retention times of 8.18, 8.33, and 9.72 minutes. LC/MS analysis indicated that the first and last of these products were 2-(4-methylphenylsulfonamido) thiazole-5-carboxylic acid, having the expected molecular weight of 272 (m/z of 273.0 and 273.0).

### Example 7

### Synthesis of 2-(5-fluorothiazol-2-yl) isoindoline-1,3-dione

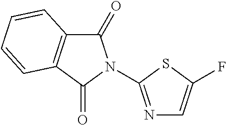 [SMILES: O=C1c2ccccc2C(=O)N1c1ncc(F)s1]

2-(2-Carboxybenzamido)thiazole-5-carboxylic acid, 0.80 g, 2.9 mmol, and F-TEDA, 1.65 g, 1.6 eq., were combined with 12 mL of methyl tetrahydrofuran in a 50 mL, 3-necked round-bottomed flask fitted with a mechanical agitator. Potassium phosphate, tribasic monohydrate, 1.87 g, 2.8 eq., was dissolved in 7 mL of water and this solution was added dropwise to the reaction over about 15 minutes while maintaining the reaction contents at 2-5° C. The ice bath was removed and the mixture was stirred vigorously at ambient temperature for four hours. Solids were removed, rinsed with methyl tetrahydrofuran, and discarded. The phases were separated and the aqueous layer was extracted with 15 mL of methyl tetrahydrofuran and the combined organic layers were washed with 15 mL of dilute aqueous sodium chloride and dried over sodium sulfate. Evaporation of the solvent provided 0.28 g, 39%, of a red-brown solid as the expected product, 2-(5-fluorothiazol-2-yl) isoindoline-1,3-dione.

1-15. (canceled)

16. A method for producing a compound represented by the formula (I): 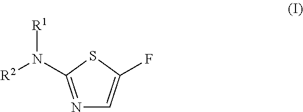
or a salt thereof, wherein R¹ and R² are the same or different and each is selected from the group consisting of a hydrogen atom, an optionally substituted carbonyl group, an optionally substituted sulfonyl group, and an optionally substituted phosphoryl group, the method comprising reacting a compound represent by the formula (II): 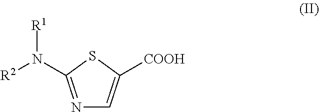
or a salt thereof with a fluorine donor.

17. The method according to claim 16, wherein each of R¹ and R² is a hydrogen atom.

18. The method according to claim 16, wherein R¹ is a hydrogen atom and R² is an optionally substituted carbonyl group which forms a carbamate with the adjacent nitrogen.

19. The method according to claim 16, wherein R¹ is a hydrogen atom and R² is an optionally substituted carbonyl group which forms an amide with the adjacent nitrogen.

20. The method according to claim 16, wherein each of R¹ and R² is an optionally substituted carbonyl group which forms an amide with the adjacent nitrogen.

21. The method according to claim 20, wherein R¹ and R² are taken together to form isoindoline 1,3-dione.

22. The method according to claim 16, wherein R¹ and R² are the same or different and each is selected from the group consisting of a hydrogen atom, an optionally substituted C₁₋₆ alkoxy-carbonyl, an optionally substituted C₁₋₆ alkyl-carbonyl, an optionally substituted sulfonyl, and an optionally substituted phosphoryl.

23. The method according to claim 22, wherein R¹ is a hydrogen atom and R² is not a hydrogen atom.

24. The method according to claim 16, wherein R¹ and R² are the same or different and each is selected from the group consisting of a hydrogen atom, tert-butoxycarbonyl, benzyloxycarbonyl, methylcarbonyl, diphenoxyphosphoryl, and 4-methylphenylsulfonyl.

25. The method according to claim 24, wherein R¹ is a hydrogen atom and R² is not a hydrogen atom.

26. The method according to claim 16, wherein R¹ is a hydrogen atom and R² is C₁₋₆ alkyl-carbonyl which is substituted by optionally substituted phenyl.

27. The method according to claim 16, wherein the fluorine donor is 1-Chloromethyl-4-fluoro-1,4-diazoniabicyclo[2.2.2]octane bis(tetrafluoroborate).

28. The method according to claim 16, wherein the fluorine donor is 1-fluoro-4-hydroxy-1,4-diazoniabicyclo[2.2.2]octane bis(tetrafluoroborate).

29. The method according to claim 16, wherein the compound represented by the formula (II) is produced by hydrolysis of an alkyl ester thereof.

30. The method according to claim 29, wherein the alkyl ester is methyl ester.

31. A method for producing a compound represented by the formula (III): 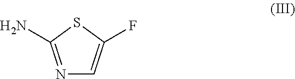
or a salt thereof comprising the steps of:
(a) reacting a compound represent by the formula (IIa): 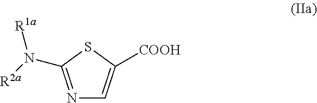
or a salt thereof with a fluorine donor under conditions that form the compound represented by the formula (I), wherein R^{1a} and R^{2a} are the same or different and each is selected from the group consisting of an optionally substituted carbonyl group, an optionally substituted sulfonyl group and an optionally substituted phosphoryl group, and
(b) deprotecting the amine of formula (I) to produce the compound of formula (III).
